(19) United States
(12) Patent Application Publication
(10) Pub. No.: US 2008/0058710 A1
(43) Pub. Date: Mar. 6, 2008

(54) Surgical kit, closure device, and associated method

(76) Inventor: Peter J. Wilk, New York, NY (US)

Correspondence Address: COLEMAN SUDOL SAPONE, P.C., 714 COLORADO AVENUE, BRIDGE PORT, CT, 06605-1601, US

(21) Appl. No.: 11/891,621

(22) Filed: Aug. 10, 2007

Related U.S. Application Data
(60) Provisional application No. 60/837,867, filed on Aug. 15, 2006.

(51) Int. Cl. A61M 1/00; A61M 29/00; A61M 31/00; A61M 5/315

(52) U.S. Cl. 604/27; 604/218; 604/60; 604/96.01

(57) ABSTRACT

A surgical device includes a tubular member, a ring made of flexible material, a resilient membrane, and a pusher member. The ring is disposed in a collapsed insertion configuration inside the tubular member and has an expanded configuration surrounding or defining an aperture, the resilient membrane being connected to the ring along an entire circumference thereof so that the membrane is at least coextensive with the aperture, thereby closing or blocking the aperture. The membrane is also disposed in a folded configuration inside the tubular member. The pusher member extends into the tubular member from a proximal end thereof for ejecting the collapsed ring and the folded membrane from a distal end of the tubular member. The ring has internal spring forces or stresses tending to open the ring from the collapsed insertion configuration to the expanded configuration upon an ejection of the ring from the tubular member.

### CROSS-REFERENCE TO RELATED APPLICATION

This application claims the benefit of U.S. Provisional Patent Application No. 60/837,867 filed Aug. 15, 2007.

### BACKGROUND OF THE INVENTION

This invention relates to medical procedures carried out without the formation of an incision in a skin surface of the patient.

Such procedures are described in U.S. Pat. Nos. 5,297,536 and 5,458,131.

As described in those patents, a method for use in intra-abdominal surgery comprises the steps of (a) inserting an incising instrument with an elongate shaft through a natural body opening into a natural body cavity of a patient, (b) manipulating the incising instrument from outside the patient to form a perforation in an internal wall of the natural internal body cavity, and (c) inserting a distal end of an elongate surgical instrument through the natural body opening, the natural body cavity and the perforation into an abdominal cavity of the patient upon formation of the perforation. Further steps of the method include (d) inserting a distal end of an endoscope into the abdominal cavity, (e) operating the surgical instrument to perform a surgical operation on an organ in the abdominal cavity, (f) viewing the surgical operation via the endoscope, (g) withdrawing the surgical instrument and the endoscope from the abdominal cavity upon completion of the surgical operation, and (h) closing the perforation.

Visual feedback may be obtained as to position of a distal end of the incising instrument prior to the manipulating thereof to form the perforation. That visual feedback may be obtained via the endoscope or, alternatively, via radiographic or X-ray equipment.

The abdominal cavity may be insulated prior to the insertion of the distal end of the endoscope into the abdominal cavity. Insufflation may be implemented via a Veress needle inserted through the abdominal wall or through another perforation in the internal wall of the natural body cavity. That other perforation is formed by the Veress needle itself. U.S. Pat. No. 5,209,721 discloses a Veress needle that utilizes ultrasound to detect the presence of an organ along an inner surface of the abdominal wall.

A method in accordance with the disclosures of U.S. Pat. Nos. 5,297,536 and 5,458,131 comprises the steps of (i) inserting an endoscope through a natural body opening into a natural body cavity of a patient, (ii) inserting an endoscopic type incising instrument through the natural body opening into the natural body cavity, (iii) manipulating the incising instrument from outside the patient to form a perforation in an internal wall of the natural internal body cavity, (iv) moving a distal end of the endoscope through the perforation, (v) using the endoscope to visually inspect internal body tissues in an abdominal cavity of the patient, (vi) inserting a distal end of an elongate surgical instrument into the abdominal cavity of the patient, (vii) executing a surgical operation on the internal body tissues by manipulating the surgical instrument from outside the patient, (viii) upon completion of the surgical operation, withdrawing the surgical instrument and the endoscope from the abdominal cavity, (ix) closing the perforation, and (x) withdrawing the endoscope from the natural body cavity.

The surgical procedures of U.S. Pat. Nos. 5,297,536 and 5,458,131 reduce trauma to the individual even more than laparoscopic procedures. Hospital convalescence stays are even shorter. There are some potential problems with the procedures, such as the difficulty in forming a fluid tight closure of the perforation formed in the wall of the hollow internal body organ.

### OBJECTS OF THE INVENTION

It is an object of the present invention to provide improvements on the afore-described surgical procedures.

It is a more specific object of the present invention to provide a method for closing a perforation or opening in an organ wall, particularly where the organ wall is located in the abdominal cavity or other body space.

A further object of the present invention is to provide a device and/or a surgical kit useful in performing the closure method.

These and other objects of the present invention will be apparent from the drawings and detailed descriptions herein. While every object of the invention is believed to be attained in at least one embodiment of the invention, there is not necessarily any single embodiment that achieves all of the objects of the invention.

### SUMMARY OF THE INVENTION

A surgical closure kit in accordance with the present invention comprises a body member with an elastic membrane attached thereto, a tubular member, a pusher member, and an at least partially solid membrane deformation element engageable with the membrane and disposable together with the body member and the membrane in a patient for deforming the membrane from a relaxed configuration to an expanded configuration and for maintaining the membrane in the expanded configuration. The body member with the membrane is initially disposed (at the onset of a surgical closure procedure) in a collapsed or folded configuration inside the tubular member. The pusher member is insertable into the tubular member for ejecting the body member together with the membrane from the tubular member.

Pursuant to further features of the present invention, the body member is a ring and the membrane is a sheet or film member connected at a periphery about an entire circumferential extent of the ring, an elongate tubular deployment member is provided for advancing the deformation element inside a patient to a surgical site, and the membrane deformation element is taken from a group consisting of a wire, a foam composition, a balloon, and liposuction material.

A more specific embodiment of a surgical closure kit comprises, in accordance with the present invention, (a) a flexible ring with a membrane attached thereto about an entire circumferential extent of the ring, and (b) a tubular member. The ring and the membrane are disposed in a collapsed or folded configuration inside the tubular member. The ring has internal spring forces or stresses tending to open the ring from the collapsed or folded configuration to an expanded configuration upon an ejection of the ring and the membrane from the tubular member. The surgical closure kit further comprises (c) a pusher member insertable into the tubular member for ejecting the ring together with the membrane from the tubular member, and (d) a membrane deformation element engageable with the membrane for deforming the membrane from a relaxed configuration substantially coplanar with the expanded configuration of the ring to a mushroom-head configuration substantially displaced from a plane of the expanded configuration of the ring.

Concomitantly, a surgical device in accordance with the present invention comprises (i) a tubular member, a ring made of flexible material, the ring being disposed in a collapsed insertion configuration inside the tubular member, the ring having an expanded configuration surrounding or defining an aperture, (ii) a resilient membrane connected to the ring along an entire circumference thereof so that the membrane is at least coextensive with the aperture, thereby closing or blocking the aperture, the membrane being disposed in a folded configuration inside the tubular member, and (iii) a pusher member extending into the tubular member from a proximal end thereof for ejecting the collapsed ring and the folded membrane from a distal end of the tubular member, the ring having internal spring forces or stresses tending to open the ring from the collapsed insertion configuration to the expanded configuration upon an ejection of the ring from the tubular member.

The membrane deformation elements may be a wire, a foam composition, liposuction material, a balloon, etc.

Pursuant to another feature of the present invention, the ring is provided with at least one pressure sensor for measuring mucosal pressure upon installation of the ring and expansion or deformation of the membrane. Also, the ring and/or the membrane may be provided with a growth factor taken from the group consisting of angiogenesis factors, mucosal implants, mucosal stimulation factors, gene therapy factors, and ox fascia.

A surgical closure method in accordance with the present invention comprises (1) inserting a distal end of a tubular member into a patient, (2) thereafter ejecting from a distal end of the tubular member a flexible ring with a membrane attached thereto about an entire circumferential extent of the ring, (3) thereafter expanding or unfolding the ring from a collapsed or folded configuration to an expanded substantially planar configuration, (4) positioning the expanded ring against a wall of an internal organ of the patient about a hole in the organ wall so that the membrane covers the hole, and (5) pressing the membrane to deform the membrane from a relaxed configuration substantially coplanar with the expanded configuration of the ring to a mushroom-head configuration substantially displaced from a plane of the expanded configuration of the ring, thereby sandwiching, between the ring and the deformed membrane, a portion of the organ wall about the hole, closing and sealing the hole.

The pressing of the membrane includes inserting a deformation element into the patient and placing the deformation element in contact with the membrane.

The membrane deformation element may be a wire. In that case, the wire may be connected at a distal end to the ring. Pushing the wire at a proximal end thereof causes the wire to deform or bend upon encountering a resistance offered by the membrane. Continuous pushing of the wire causes the wire to curl in a tangled ball and push the membrane into a ballooned or mushroom configuration, with a rim of organ wall along the opening being sandwiched between the expanded membrane and the ring. The wire is preferably introduced into the patient through a catheter, so that bending of the wire occurs only at the membrane and not along an insertion path of the instrumentation.

The membrane deformation element may alternatively take the form of a foam composition. The foam composition should not only be biocompatible, but also impervious to a low acid environment where the opening to be closed is in a gastric wall. In addition, the foam composition may be quick hardening, in which case injection pressure of the foam may be sufficient to expand the membrane and hold the membrane in the deformed bulbous or mushroom head shape. Alternatively, a balloon may be provided for temporarily deforming the membrane to facilitate a filling of the deformed membrane with the foam composition. In that event, upon expansion of the balloon to push against the membrane, the foam is ejected behind the expanded balloon, that is between the balloon and the membrane. The balloon is deflated as the foam is dispensed, so that the foam takes the place of the balloon.

The membrane deformation element may be liposuction material. Preferably, the liposuction material is harvested from the patient prior to implantation of the ring and membrane.

Where the ring is provided with at least one pressure sensor, the method further comprises operating the sensor to measure mucosal pressure upon installation of the expanded ring and expansion or deformation of the membrane. The pressure feedback is used to ensure that the gripping of the organ wall between the expanded membrane and the ring is sufficiently tight to seal the hole or opening in the organ wall, but not so tight as to cause tissue necrosis.

As indicated above, the ring and/or the membrane may be provided with a growth factor taken from the group consisting of angiogenesis factors, mucosal implants, mucosal stimulation factors, gene therapy factors, and ox fascia. More generally, a surgical device may comprise an implantable body having at least one surface disposable in contact with internal tissues of a patient upon implantation of the body, at least part of the body being provided with a growth factor taken from the group consisting of angiogenesis factors, mucosal implants, mucosal stimulation factors, gene therapy factors, and ox fascia.

### BRIEF DESCRIPTION OF THE DRAWINGS

FIG. 1 is a top plan view of a surgical closure device in accordance with the present invention.

FIG. 2 is a cross-sectional view, on a slightly larger scale, taken along line II-II in FIG. 1.

FIG. 3 is a schematic longitudinal cross-sectional view, on a substantially enlarged scale, of a deployment tube carrying the device of FIGS. 1 and 2 in a collapsed configuration.

FIG. 4 is a schematic cross-sectional view of the surgical closure device of FIGS. 1 and 2, showing the device in a deformed and filled use configuration.

FIG. 5 is a schematic side elevational view of a deployment tube for dispensing a membrane deformation agent against or into the surgical device of FIGS. 1 and 2.

FIGS. 6A-6E are schematic cross-sectional views of a stomach, showing successive steps in an endoscopic surgical closure procedure in accordance with the present invention.

FIG. 7 is a schematic cross-sectional view, similar to FIG. 4, depicting a method of membrane deformation utilizable with the device of FIGS. 1 and 2.

FIG. 8 is a schematic cross-sectional view, similar to FIG. 7, depicting a modified method of membrane deformation utilizable with the device of FIGS. 1 and 2.

FIG. 9 is a schematic cross-sectional view, similar to FIG. 7, depicting another method of membrane deformation utilizable with the device of FIGS. 1 and 2.

FIG. 10 is a schematic cross-sectional view, similar to FIG. 2, illustrating a modified embodiment of a surgical closure device in accordance with the present invention.

FIG. 11 is a schematic cross-sectional view Similar to FIGS. 7-9, showing a step in the deployment and deformation of the device of FIG. 9.

### DETAILED DESCRIPTION

As shown in FIGS. 1 and 2, a surgical closure device 12 comprises a ring 14 made of flexible material and an elastic membrane 16 attached about its periphery to the ring along the entire circumferential extent thereof, thereby covering an aperture 17 defined by the ring. Membrane 16 is at least coextensive with aperture 17, thereby closing or blocking the aperture. Both ring 14 and membrane 16 are made of a biocompatible polymeric material that is acid stable or impervious, thereby enabling implantation of the closure into the stomach for covering a gastric perforation formed, for instance, during a trans-organ or incisionless surgery as described in U.S. Pat. Nos. 5,297,536 and 5,458,131.

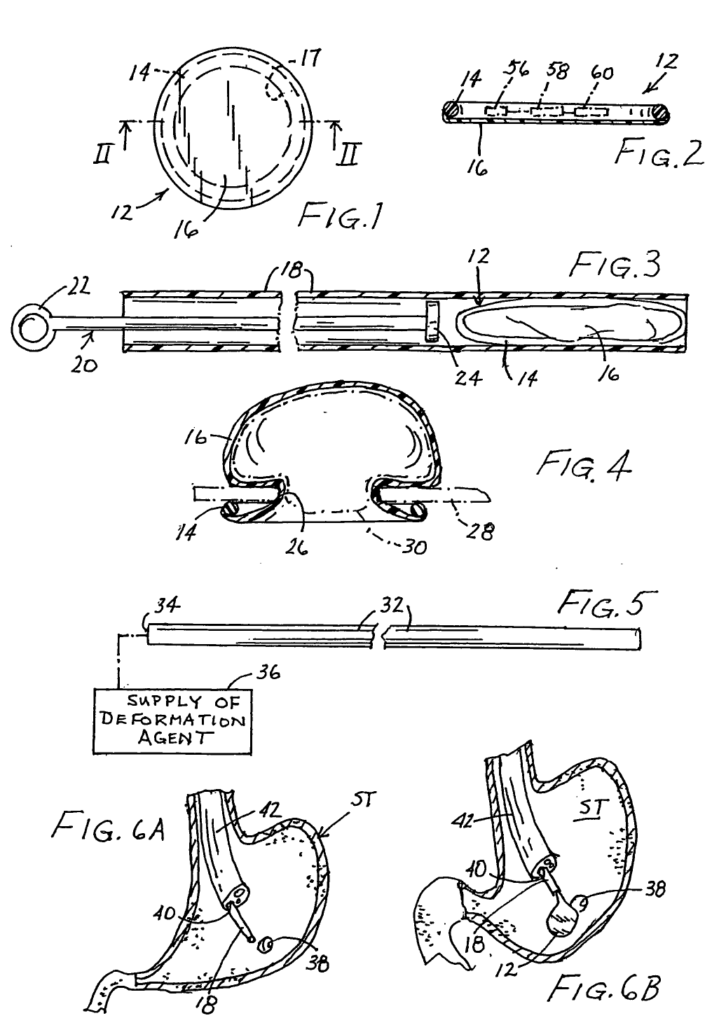

Ring 14 and membrane 16 may be inserted into a hollow internal organ of a patient, such as the stomach, the vagina, the urinary bladder or the colon, by means of a tubular deployment member 18 as illustrated in FIG. 3. Deployment member 18 is made of a partially flexible material where closure device 12 is to be implanted, for example, in the stomach or colon. The flexibility of deployment member 18 enables that member to negotiate curves and bends along an insertion pathway such as the mouth and esophagus or the sigmoidal colon.

During an insertion operation, closure device 12, particularly including ring 14 and membrane 16, is disposed in a collapsed insertion configuration inside tubular deployment member 18, as depicted in FIG. 3. A pusher member 20 including a handle 22 and a pusher plate 24 is insertable into deployment member 18 for ejecting closure device 12 from the deployment member upon the attainment of a desired surgical site inside a patient.

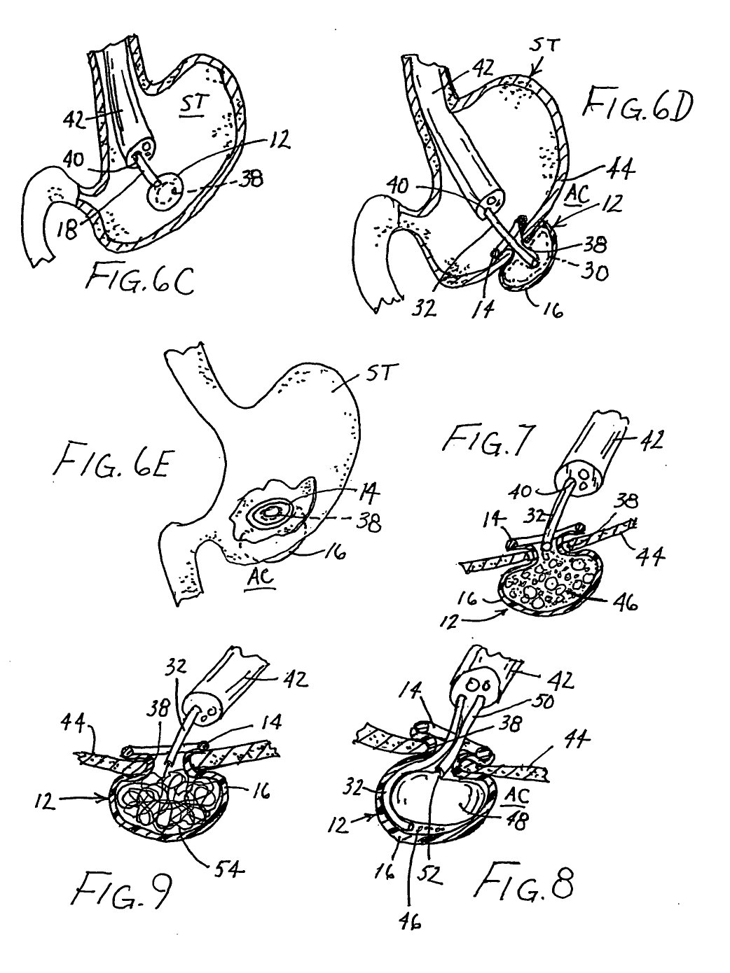

FIG. 4 illustrates closure device 12 in an expanded configuration closing an opening or perforation 26 in a wall 28 of an internal organ. More particularly, membrane 16 is deformed from a planar relaxed or neutral configuration shown in FIG. 2 to a bulbous or mushroom-shaped clamping and plugging configuration shown in FIG. 4. Membrane 16 is deformed and held in the deformed configuration by a deformation agent or element 30. Deformation agent or element 20 is inserted into the patient and ejected against membrane 16 via a deployment tube 32 operatively connected at a proximal end 34 to a supply 36 of the deformation agent (FIG. 5). The deformation agent 30 is made of an at least partially solid material that remains inside the patient together with closure device 12 upon completion of a surgical closure procedure. Such a procedure is depicted in FIGS. 6A-6E.

As shown in FIG. 6A, a patient's stomach ST has a perforation 38 formed, for instance, during intra-abdominal surgery conducted at least partially via perforation 38. At the end of the intra-abdominal procedure, a distal end portion of tubular deployment member 18 is inserted into stomach ST via a biopsy or working channel 40 of an endoscope 42 in turn inserted into the stomach via the patient's esophagus ES. Pusher member 20 is shifted in the distal direction, towards perforation 38, thereby ejecting closure device 12, as shown in FIG. 6B. Upon ejection from the distal end of tubular deployment member 18, ring 14 expands automatically in response to internal spring forces to assume a circular configuration as shown in FIGS. 1 and 2.

The expanded or unfolded closure device 12 is placed in contact with a wall 44 of stomach ST so that ring 14 surrounds perforation 38 and membrane 16 covers the perforation, as depicted in FIG. 6C. If necessary, an endoscopic forceps (not shown) may be inserted via endoscope 42 either via working channel 40 or via a second biopsy or working channel (not shown) and manipulated to grasp the expanded closure device 12 and position it over perforation 38.

After the placement of closure device 12 over perforation 38, a distal end portion (not separately labeled) of deployment tube 32 is inserted into the patient via endoscope 42, as shown in FIG. 6C. Deployment tube 32 and supply 36 is operated extracorporeally to eject deformation agent 34 against membrane 16, thereby forcing the membrane to expand into the patient's abdominal cavity AC. The distal tip (not separately designated) may be inserted through ring 14 and into the expanded membrane 16, as shown in FIG. 6D, to facilitate a filling of the membrane 16. The deformed membrane forms a bulbous pouch (FIGS. 4, 6D, 6E) similar in shape to a mushroom head.

Deformation agent 34 may particularly take the form of a foam composition 46, as depicted in FIG. 7. The foam composition 46 should not only be biocompatible, but also impervious to a low acid environment inasmuch as closure device 12 will be subject to gastric juices. This same requirement applies to the materials of ring 14 and membrane 16. This materials requirement will not necessarily pertain if closure device 12 is used to plug openings formed in other hollow internal organs such as the vagina or the urinary bladder.

Foam composition 46 may be ejected from deployment tube 32 at such a rate that the force of the foam composition against membrane 16 is sufficient to distort the membrane. Preferably, foam composition 46 is quick hardening, so that membrane 16 is held in the expanded bulbous, enlarged or mushroom-head shape.

In an alternative technique depicted in FIG. 8, a balloon 48 is used to expand and hold membrane 16 in an expanded configuration while foam composition 46 is injected via deployment tube 32 behind the expanded balloon 48, that is, between the balloon and the membrane. During insertion into the patient via working channel 40 of endoscope 42, balloon 48 is housed in a collapsed configuration inside a distal end portion of a tubular deployment member 50. After closure device 12 has been positioned over perforation 38 and after an extension of the distal end portion of deployment member 50 from working channel 40 inside the patient's stomach, balloon 48 is ejected and partially inflated to push membrane 16 out of the plane of ring 14 and perforation 38 and into the patient's abdominal cavity AC. During this initial pushing against membrane 16, the inflated balloon 48 is smaller in diameter than the diameter of perforation 38, so that balloon 48 together with membrane 16 may pass through the perforation into the patient's abdominal cavity AC. Thereafter, balloon 48 is inflated further so that membrane 16 has a form significantly larger than perforation 38, large enough to clamp and seal the edges or periphery (not separately designated) of perforation. As foam composition 46 is ejected from deployment tube 32 into a space between balloon 48 and membrane 16 and hardens, balloon 48 may be deflated and ultimately withdrawn through perforation 38 and out of the patient.

In an alternative procedure, balloon 48 by itself may serve as a permanent deformation agent, not only deforming membrane 16 but also holding the membrane in the expanded or enlarged bulbous configuration. In that case, a neck 52 of balloon 48 is clamped, crimped, tied, sutured, glued, heat sealed, ultrasonically welded or otherwise closed to maintain balloon 48 in an inflated configuration larger in diameter than perforation 38. To that end, a distal end portion of an ancillary instrument (not shown) may be inserted into the patient's stomach through a working channel of endoscope 42, or separately alongside endoscope 42 or via a channel in a sheath encasing endoscope 42. Again, in the case of gastric perforation 38, the material of balloon 48 should not only be elastic and biocompatible, but also impervious to a low acid environment.

The foam composition 46 of FIG. 8 could be replaced by a liposuction material such as fragmented adipose tissue. In that case, the liposuction composition is preferably but not necessarily obtained from the same patient prior to the trans-organ surgery. Alternatively, the liposuction material may be obtained from a compatible human or animal donor.

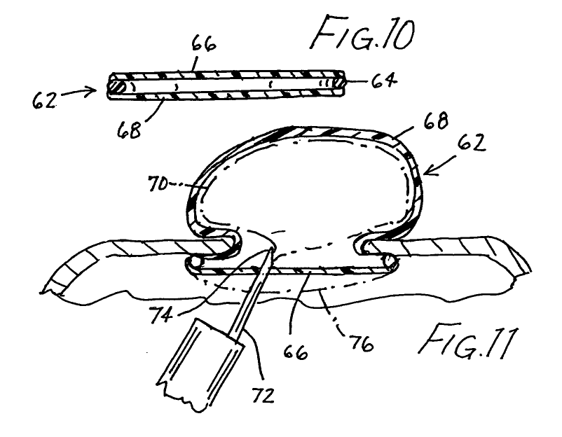

Deformation agent 34 may alternatively take the form of a wire element 54, as depicted in FIG. 9. Wire 54 may be connected at a distal end to ring 14. Pushing wire 54 from a proximal end thereof causes the wire to deform or bend upon encountering a resistance offered by membrane 16. Continuous pushing of wire 54 causes the wire to curl in a tangled ball as shown in FIG. 9 and push the membrane into an expanded, enlarged, ballooned or mushroomed configuration, with a rim of organ wall 44 along the perforation 38 being sandwiched between the expanded membrane 16 and ring 14. Wire 54 is preferably introduced into the patient through deployment tube 32 such as a catheter, so that bending of the wire occurs only at the membrane 16 and not along an insertion path of the instrumentation.

Ring 12 may be provided with at least one pressure sensor 56, a wireless transmitter 58 and a battery 60. Sensor 56 functions to measure mucosal pressure upon installation of the expanded ring 14 and expansion or deformation of membrane 16. The pressure feedback is used by a surgeon or other medical practitioner to ensure that the gripping of the organ wall 44 between the expanded membrane 16 and the ring 14 is sufficiently tight to seal the perforation or opening 38 in organ wall, but not so tight as to cause tissue necrosis.

As shown in FIG. 10, an alternative closure device 62 comprises a ring 64 of flexible material and two membranes 66 and 68 disposed on opposite sides or the same side of ring 62. One membrane 66 may be substantially stiffer than then other membrane 68 so that disposition of a deformation agent or element 70 between membranes 66 and 68 during a closure procedure as described above causes deformation mostly of membrane 68 and not membrane 66. In that case, membrane 66 functions more in the nature of a flexible disk (collapsed during insertion) rather than an elastic membrane. A deployment tube 72 for the insertion of deformation agent or element 70 between membranes 66 and 68 may be provided with a sharp distal tip 74 for piercing membrane 66. Alternatively, membrane 66 and/or ring 62 may be provided with a valve (not shown) connectable with the deployment tube for receiving and passing the deformation agent or element 70. FIG. 11 shows in phantom lines 76, a deformed configuration for membrane 66.

Although the invention has been described in terms of particular embodiments and applications, one of ordinary skill in the art, in light of this teaching, can generate additional embodiments and modifications without departing from the spirit of or exceeding the scope of the claimed invention. Accordingly, it is to be understood that the drawings and descriptions herein are profferred by way of example to facilitate comprehension of the invention and should not be construed to limit the scope thereof.

What is claimed is:

1. A surgical closure kit comprising:
a body member with at least one elastic membrane attached thereto;
a tubular member, said body member with said membrane being disposed in a collapsed or folded configuration inside said tubular member;
a pusher member insertable into said tubular member for ejecting said body member together with said membrane from said tubular member; and
an at least partially solid membrane deformation element engageable with said membrane and disposable together with said body member and said membrane in a patient for deforming said membrane from a relaxed configuration to an expanded configuration and for maintaining said membrane in said expanded configuration.

2. The surgical closure kit defined in claim 1 wherein said membrane deformation element is taken from a group consisting of a wire, a foam composition, a balloon, and liposuction material.

3. The surgical closure kit defined in claim 1 wherein said body member is a ring and said membrane is a sheet or film member connected at a periphery about an entire circumferential extent of said ring.

4. The surgical closure kit defined in claim 1, further comprising an elongate tubular deployment member for advancing said deformation element inside a patient to a surgical site.

5. A surgical closure kit comprising:
a flexible ring with at least one membrane attached thereto about an entire circumferential extent of said ring;
a tubular member, said ring with said membrane being disposed in a collapsed or folded configuration inside said tubular member, said ring having internal spring forces or stresses tending to open said ring from said collapsed or folded configuration to an expanded configuration upon an ejection of said ring and said membrane from said tubular member;
a pusher member insertable into said tubular member for ejecting said ring together with said membrane from said tubular member; and
a membrane deformation element engageable with said membrane for deforming said membrane from a relaxed configuration substantially coplanar with said expanded configuration of said ring to a mushroom-head configuration substantially displaced from a plane of said expanded configuration of said ring.

6. The surgical closure kit defined in claim 5 wherein said membrane deformation element is taken from a group consisting of a wire, a foam composition, a balloon, and liposuction material.

7. The surgical closure kit defined in claim 6 wherein said membrane deformation element is a wire, said wire being connected at a distal end to said ring.

8. The surgical closure kit defined in claim 6 wherein said membrane deformation element is a foam composition, further comprising a balloon for temporarily deforming said membrane to facilitate a filling of the deformed membrane with said foam composition.

9. The surgical closure kit defined in claim 6 wherein said membrane deformation element is liposuction material harvested from a patient in which said ring and membrane are implanted.

10. The surgical closure kit defined in claim 5 wherein said ring is provided with at least one pressure sensor for measuring mucosal pressure upon installation of said ring and expansion or deformation of said membrane.

11. The surgical closure kit defined in claim 5 wherein at least one of said ring and said membrane is provided with a growth factor taken from the group consisting of angiogenesis factors, mucosal implants, mucosal stimulation factors, gene therapy factors, and ox fascia.

12. A surgical device comprising:
a tubular member;
a ring made of flexible material, said ring being disposed in a collapsed insertion configuration inside said tubular member, said ring having an expanded configuration surrounding or defining an aperture;
at least one resilient membrane connected to said ring along an entire circumference thereof so that said membrane is at least coextensive with said aperture, thereby closing or blocking said aperture, said membrane being disposed in a folded configuration inside said tubular member; and
a pusher member extending into said tubular member from a proximal end thereof for ejecting the collapsed ring and the folded membrane from a distal end of said tubular member, said ring having internal spring forces or stresses tending to open said ring from said collapsed insertion configuration to said expanded configuration upon an ejection of said ring from said tubular member.

13. The surgical device defined in claim 12 wherein said ring is provided with at least one pressure sensor for measuring mucosal pressure upon installation of said ring and expansion or deformation of said membrane.

14. The surgical closure kit defined in claim 12 wherein at least one of said ring and said membrane is provided with a growth factor taken from the group consisting of angiogenesis factors, mucosal implants, mucosal stimulation factors, gene therapy factors, and ox fascia.

15. A surgical closure method comprising:
inserting a distal end of a tubular member into a patient;
thereafter ejecting from a distal end of said tubular member a flexible ring with at least one membrane attached thereto about an entire circumferential extent of said ring;
upon ejecting of said ring and said membrane, expanding or unfolding said ring from a collapsed or folded configuration to an expanded substantially planar configuration;
positioning the expanded ring against a wall of an internal organ of the patient about a hole in the organ wall so that the membrane covers the hole; and
pressing said membrane to deform said membrane from a relaxed configuration substantially coplanar with said expanded configuration of said ring to a mushroom-head configuration substantially displaced from a plane of said expanded configuration of said ring, thereby sandwiching, between said ring and the deformed membrane, a portion of the organ wall about the hole, closing and sealing the hole.

16. The method defined in claim 15 wherein the pressing of the membrane includes inserting a deformation element into the patient and placing said deformation element in contact with said membrane.

17. The method defined in claim 16 wherein said deformation element is a wire, the inserting of said deformation element including bending said wire multiple times.

18. The method defined in claim 16 wherein said membrane deformation element is a foam composition, further comprising inserting a foam ejector into the patient and dispensing said foam from a distal end of said foam ejector against said membrane.

19. The method defined in claim 16 wherein said membrane deformation element is liposuction material harvested from a patient in which said ring and membrane are implanted, further comprising inserting an ejector instrument into the patient and dispensing said foam from a distal end of said ejector instrument against said membrane.

20. The method defined in claim 15 wherein said ring is provided with at least one pressure sensor, further comprising operating said sensor to measure mucosal pressure upon installation of the expanded ring and expansion or deformation of said membrane.

21. The method defined in claim 15 wherein at least one of said ring and said membrane is provided with a growth factor taken from the group consisting of angiogenesis factors, mucosal implants, mucosal stimulation factors, gene therapy factors, and ox fascia.

22. A surgical device comprising an implantable body having at least one surface disposable in contact with internal tissues of a patient upon implantation of said body, at least part of said body being provided with a growth factor taken from the group consisting of angiogenesis factors, mucosal implants, mucosal stimulation factors, gene therapy factors, and ox fascia.
